(19) United States
(12) Patent Application Publication
(10) Pub. No.: US 2022/0126290 A1
(43) Pub. Date: Apr. 28, 2022

(54) MICRODEVICE, MANUFACTURING METHOD FOR MICRODEVICE, AND IMMUNOASSAY METHOD

(71) Applicant: TIANMA JAPAN, LTD., Kawasaki (JP)

(72) Inventors: Chikaaki MIZOKUCHI, Kawasaki (JP); Koji Shigemura, Kawasaki (JP); Ken Sumiyoshi, Kawasaki (JP)

(73) Assignee: TIANMA JAPAN, LTD., Kawasaki (JP)

(21) Appl. No.: 17/503,648

(22) Filed: Oct. 18, 2021

(30) Foreign Application Priority Data
Oct. 23, 2020 (JP) 2020-178217

(51) Int. Cl. B01L 3/00; G01N 33/58

(52) U.S. Cl. CPC B01L 3/502715; B01L 3/502707; G01N 33/53; B01L 2200/0689; B01L 2300/165; G01N 33/582

(57) ABSTRACT

A microdevice includes a plurality of calibration curve liquids, a plurality of first microchannels respectively filled with the plurality of calibration curve liquids, at least one second microchannel filled with a measurement target liquid, and a sealing member that closes openings of the first microchannels to seal the calibration curve liquids. Each of the calibration curve liquids includes a measurement target substance of a predetermined concentration, the predetermined concentration in each of the plurality of calibration curve liquid being mutually different, an antibody that specifically binds to the measurement target substance, and a fluorescent-labeling derivative that fluorescently labels the measurement target substance and competes with the measurement target substance to specifically bind to the antibody. The measurement target liquid includes an unknown concentration of the measurement target substance, the antibody, and the fluorescent-labeling derivative.

### CROSS-REFERENCE TO RELATED APPLICATION

This application claims the benefit of Japanese Patent Application No. 2020-178217, filed on Oct. 23, 2020, the entire disclosure of which is incorporated by reference herein.

### FIELD

This application relates generally to a microdevice, a manufacturing method for the microdevice, and an immunoassay method.

### BACKGROUND

Among immunoassay methods that use fluorescence, fluorescence polarization immunoassay methods (FPIA) that use antigen-antibody reactions to detect a measurement target substance are known. For example, Japanese Patent Application Publication No. H03-103765 describes a method for calculating the concentration of a measurement antigen (measurement target substance) from the measured degree of polarization of fluorescence.

Additionally, immunoassay methods that use microdevices are known. For example, Japanese Patent No. 4717081 describes an immunoassay microchip in which microstructures are arranged in channels. The microstructures retain beads that have primary antibodies solidified on the surfaces thereof.

In a fluorescence polarization immunoassay method that uses a microdevice having a plurality of channels, it is possible to improve measurement reliability by measuring, at once, a plurality of samples for creating a calibration curve (for example, a plurality of diluted liquids obtained by diluting a standard liquid in a stepwise manner) and a sample containing the measurement target substance. In this immunoassay method, each time measurement is performed, the channels are filled with the plurality of samples for creating the calibration curve. Consequently, the work prior to starting the measurement of the degree of polarization increases.

### SUMMARY

A microdevice according to a first aspect of the present disclosure includes:

a plurality of calibration curve liquids including a measurement target substance of a predetermined concentration, the predetermined concentration in each of the plurality of calibration curve liquids being mutually different, an antibody that specifically binds to the measurement target substance, and a fluorescent-labeling derivative that fluorescently labels the measurement target substance and competes with the measurement target substance to specifically bind to the antibody;

a plurality of first microchannels respectively filled with the plurality of calibration curve liquids;

at least one second microchannel to be filled with a measurement target liquid including an unknown concentration of the measurement target substance, the antibody, and the fluorescent-labeling derivative; and

a sealing member that closes an opening of the first microchannels to seal the calibration curve liquids.

A manufacturing method for the microdevice according to a second aspect of the present disclosure includes:

forming a plurality of first microchannels, and at least one second microchannels to be filled with a measurement target liquid including an unknown concentration of a measurement target substance, an antibody that specifically binds to the measurement target substance, and a fluorescent-labeling derivative that fluorescently labels the measurement target substance and competes with the measurement target substance to specifically bind to the antibody;

filling the plurality of first microchannels respectively with a plurality of calibration curve liquids including the measurement target substance of a predetermined concentration, the predetermined concentration in each of the plurality of calibration curve liquid being mutually different, the antibody, and the fluorescent-labeling derivative; and

sealing the plurality of calibration curve liquids by closing, by a sealing member, an opening of the first microchannels that are filled with the calibration curve liquids.

An immunoassay method according to a third aspect of the present disclosure includes:

setting a temperature of the microdevice according to the first aspect of the present disclosure, that is stored at a temperature of 5° C. or lower, to a predetermined measurement temperature;

filling the second microchannel with the measurement target liquid;

calculating a degree of polarization of fluorescence emitted from the plurality of calibration curve liquids and the measurement target liquid with which the second microchannel is filled;

creating a calibration curve of the degree of polarization and a concentration of the measurement target substance from the calculated degree of polarization of the fluorescence emitted from the plurality of calibration curve liquids; and

calculating the concentration of the measurement target substance included in the measurement target liquid from the calculated degree of polarization of the fluorescence emitted from the measurement target liquid and the created calibration curve.

It is to be understood that both the foregoing general description and the following detailed description are exemplary and explanatory and are not restrictive of this disclosure.

### BRIEF DESCRIPTION OF THE DRAWINGS

A more complete understanding of this application can be obtained when the following detailed description is considered in conjunction with the following drawings, in which:

FIG. 1 is a top view illustrating a microdevice according to an embodiment;

FIG. 2 is a cross-sectional view of the microdevice illustrated in FIG. 1, taken along line A-A;

FIG. 3 is a schematic drawing illustrating a calibration curve liquid according to the embodiment;

FIG. 4 is a cross-sectional view illustrating a sealing member according to the embodiment;

FIG. 5 is a flowchart illustrating a manufacturing method for the microdevice according to the embodiment;

FIG. 6 is a schematic drawing for explaining a step of integrally forming a second substrate and a partition wall according to the embodiment;

FIG. 7 is a drawing illustrating the configuration of an analysis device according to the embodiment;

FIG. 8 is a schematic drawing illustrating the analysis device according to the embodiment;

FIG. 9 is a flowchart illustrating an immunoassay method according to the embodiment;

FIG. 10 is a schematic drawing for explaining filling of a measurement target liquid in the immunoassay method according to the embodiment; and

FIG. 11 is a drawing illustrating the relationship between concentration and degree of polarization according to examples and a comparative example.

### DETAILED DESCRIPTION

Hereinafter, a microdevice according to various embodiments is described while referencing the drawings.

### Embodiments

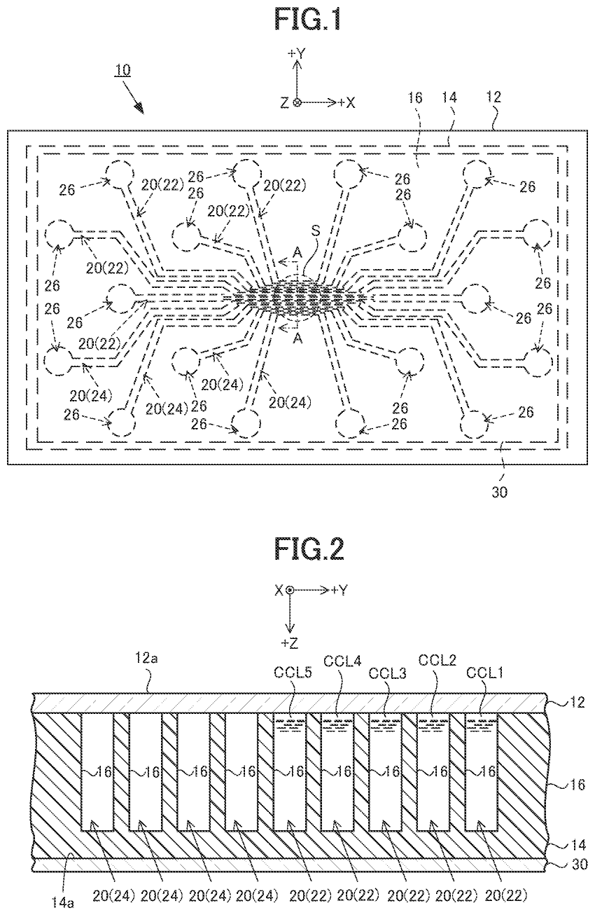

A microdevice 10 according to the present embodiment is described while referencing FIGS. 1 to 10. In one example, the microdevice 10 is used in the detection of a measurement target substance Ag1 using a fluorescence polarization immunoassay method.

As illustrated in FIGS. 1 and 2, the microdevice 10 includes a first substrate 12, a second substrate 14, a partition wall 16, nine microchannels 20, and a sealing member 30. Additionally, the microdevice 10 includes five calibration curve liquids CCL1 to CCL5 (described later). The first substrate 12, the second substrate 14, and the partition wall 16 form the microchannels 20. The sealing member 30 closes openings 26 of the microchannels 20. Five microchannels 20 of the nine microchannels 20 are respectively filled with the calibration curve liquids CCL1 to CCL5.

In the present specification, the calibration curve liquids may be referred to collectively as “calibration curve liquids CCL.” Additionally, the microchannels 20 filled with the calibration curve liquids CCL are referred to as “first microchannels 22”, and the other microchannels 20 are referred to as “second microchannels 24.” To facilitate comprehension, in the microdevice 10 of FIG. 1, the right direction (the right direction on paper) is referred to as the “+X direction”, the up direction (the up direction on paper) is referred to as the “+Y direction”, and the direction perpendicular to the +X direction and the +Y direction (the depth direction on paper) is referred to as the “+Z direction.”

The first substrate 12 of the microdevice 10 is a flat plate-like quartz glass substrate. Excitation light EL in the fluorescence polarization immunoassay method enters the microdevice 10 from the first substrate 12. The excitation light EL falls on a measurement region S illustrated in FIG. 1, and perpendicularly enters a first main surface 12a of the first substrate 12.

The second substrate 14 of the microdevice 10 is a flat plate-like substrate. The second substrate 14 is formed from a material that has little autofluorescence. In the present embodiment, the second substrate 14 is formed from carbon black-containing polydimethylsiloxane (PDMS). The second substrate 14 faces the first substrate 12. The second substrate 14 and the first substrate 12 sandwich the partition wall 16.

The partition wall 16 of the microdevice 10 is sandwiched by the first substrate 12 and the second substrate 14 to form the microchannels 20. The partition wall 16 is formed from a material that has little autofluorescence. Additionally, it is preferable that the partition wall 16 is formed from a material that absorbs light such as the excitation light EL, fluorescence FL, and the like. In the present embodiment, the partition wall 16 is formed integrally with the second substrate 14 from carbon black-containing polydimethylsiloxane.

The microchannels 20 of the microdevice 10 extend parallel to the X direction in the measurement region S. In one example, the width (that is, the length in the Y direction) of the microchannels 20 in the measurement region S is 200 pin. Each of the microchannels 20 includes two openings 26 that penetrate the second substrate 14 and the partition wall 16. The calibration curve liquids CCL or a hereinafter described measurement target liquid (MTL) are filled or discharged through the openings 26.

Of the nine microchannels 20, the five first microchannels 22 positioned on the +Y side when viewed from above are filled with the calibration curve liquids CCL. Of the nine microchannels 20, the four second microchannels 24 positioned on the −Y side when viewed from above are not filled with anything. The second microchannels 24 are filled with the measurement target liquid (MTL) prior to the measurement of the degree of polarization P in the fluorescence polarization immunoassay being performed.

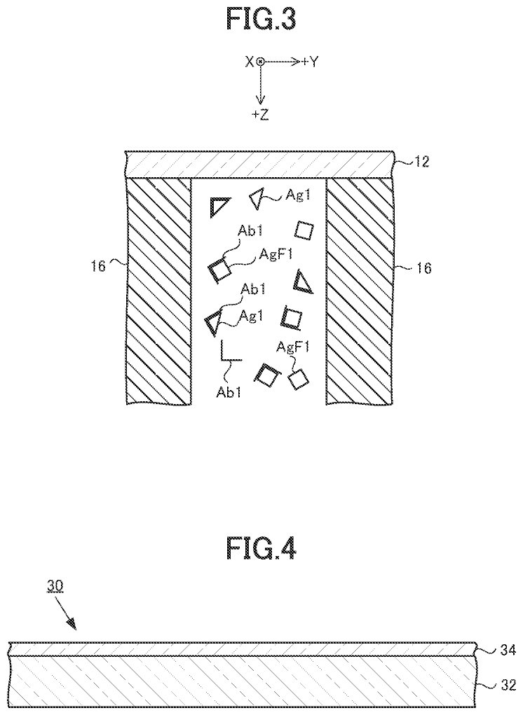

As illustrated in FIG. 3, the calibration curve liquids CCL of the microdevice 10 include the measurement target substance Ag1, an antibody Abl, and a fluorescent-labeling derivative AgF1. The calibration curve liquids CCL are used in the creation of a calibration curve (specifically, a calibration curve of the degree of polarization P and the concentration of the measurement target substance Ag1) in the fluorescence polarization immunoassay. The calibration curve can be obtained by fitting the degree of polarization P of the fluorescence FL emitted from the calibration curve liquids CCL1 to CCL5 and the concentration of the measurement target substance Ag1 in the calibration curve liquids CCL1 to CCL5 to a logistic function. In this case, it is preferable that the determination coefficient of the fitting is greater than 0.99.

The first microchannels 22 are respectively filled, through the openings 26, with the calibration curve liquids CCL1 to CCL5. The calibration curve liquids CCL1 to CCL5 each include a mutually different predetermined concentration (first concentration to fifth concentration) of the measurement target substance Ag1, a predetermined concentration (sixth concentration) of an antibody Abl, and a predetermined concentration (seventh concentration) of a fluorescent-labeling derivative AgF1.

It is sufficient that the measurement target substance Ag1 is a compound that is detectable by an immunoassay method using fluorescence. Examples of the measurement target substance Ag1 include antibiotics, bioactive substances, mold poisons, and the like. Specific examples of the measurement target substance Ag1 include prostaglandin E2, β-lactoglobulin, chloramphenicol, deoxynivalenol, and the like. In one example, the concentrations (the first concentration to the fifth concentration) of the substances to be measured Ag1 in the calibration curve liquids CCL1 to CCL5 are, respectively, 50 ng/ml, 25 ng/ml, 12.5 ng/ml, 6.25 ng/ml, and 3.125 ng/ml.

The antibody Abl specifically binds to the measurement target substance Ag1 due to an antigen-antibody reaction. In one example, the antibody Abl is obtained by inoculating a host animal (for example, a mouse or a cow) with the measurement target substance Ag1 and, then, recovering and purifying the antibodies in the blood produced by the host animal. Alternatively, a commercially available antibody can be used as the antibody Abl.

The fluorescent-labeling derivative AgF1 is a derivative that is obtained by fluorescently labeling the measurement target substance Ag1. The fluorescent-labeling derivative AgF1 competes with the measurement target substance Ag1 and specifically binds to the antibody Abl due to an antigen-antibody reaction. The fluorescent-labeling derivative AgF1 is obtained as a result of binding a fluorescent substance to the measurement target substance Ag1 using a known method. The fluorescent substance is fluorescein (wavelength of excitation light EL: 494 nm, wavelength of fluorescence FL: 521 nm), rhodamine 13 (wavelength of excitation light EL: 550 nm, wavelength of fluorescence FL: 580 nm), or the like.

Next, the measurement target liquid MTL is described. The measurement target liquid MTL is the liquid to be measured in the fluorescence polarization immunoassay. The measurement target liquid MTL includes an unknown concentration of a measurement target substance Ag1, and the same concentrations of the antibody Abl and the fluorescent-labeling derivative AgF1 as in the calibration curve liquids CCL. The second microchannels 24 are filled with the measurement target liquid MTL through the openings 26.

The sealing member 30 of the microdevice 10 is provided on a first main surface 14a of the second substrate 14, and closes the openings 26 of the microchannels 20. As illustrated in FIG. 4, the sealing member 30 includes a base material 32 and an adhesive layer 34. The base material 32 is formed from silicone resin, polyethylene terephthalate resin, or the like, for example. In one example, the adhesive layer 34 is a silicone adhesive layer. In the present embodiment, the adhesive layer 34 is adhered to the first main surface 14a of the second substrate 14 to close all of the openings 26 of the nine microchannels 20 (specifically, the five first microchannels 22 and the four second microchannels 24).

In the present embodiment, the openings 26 of the first microchannels 22 are closed by the sealing member 30, and the calibration curve liquids CCL that the first microchannels 22 are filled with are sealed in the first microchannels 22 by the sealing member 30. As a result, when the microdevice 10 is stored at a low temperature (for example, 5° C. or lower), changes over time of the calibration curve liquids CCL are suppressed. That is, with the microdevice 10, it is possible to stably store the calibration curve liquids CCL for an extended period of time at a low temperature.

Since the microdevice 10 can stably store the calibration curve liquids CCL for an extended period of time at a low temperature, it is possible to create the calibration curve and detect the measurement target substance Ag1 in the measurement target liquid MTL by simply filling the second microchannels 24 with the measurement target liquid MTL. Accordingly, by using the microdevice 10, an immunoassay can be performed with less work.

Furthermore, in the present embodiment, the openings 26 of the second microchannels 24 are also closed by the sealing member 30 and, as such, debris, impurities, and the like can be prevented from entering the second microchannels 24 that are filled with the measurement target liquid MTL.

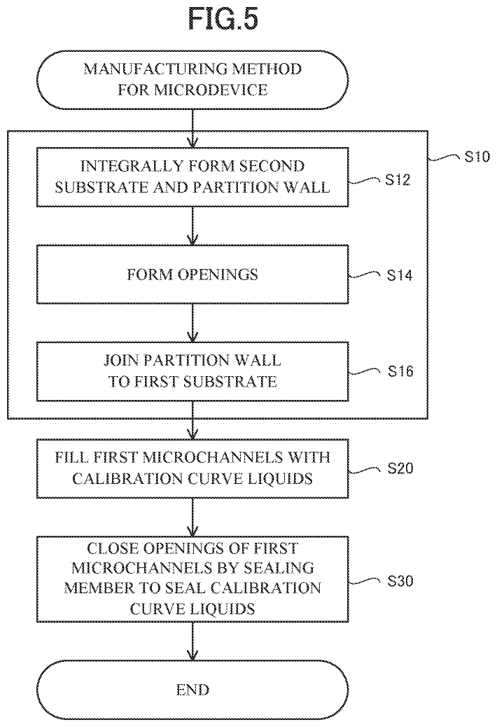

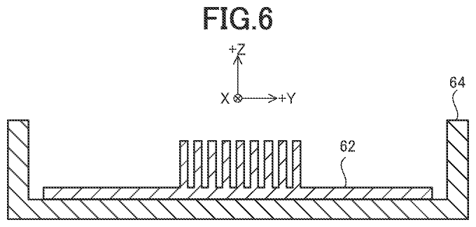

Next, a manufacturing method for the microdevice 10 is described while referencing FIGS. 5 and 6. FIG. 5 is a flowchart illustrating the manufacturing method for the microdevice 10. The manufacturing method for the microdevice 10 includes a step of forming the plurality of first microchannels 22 and at least one second microchannel 24 (step S10); a step of filling the plurality of first microchannels 22 respectively with the plurality of calibration curve liquids CCL1 to CCL5 (step S20); and a step of closing, by the sealing member 30, the openings 26 of the first microchannels 22 that are filled with the calibration curve liquids CCL1 to CCL5, thereby sealing the plurality of calibration curve liquids CCL1 to CCL5 (step S30).

As illustrated in FIG. 5, step S10 includes a step of integrally forming the second substrate 14 and the partition wall 16 (step S12), a step of forming the openings 26 (step S14), and a step of joining the partition wall 16 to the first substrate 12 (step S16).

In step S12, as illustrated in FIG. 6, a die 62, corresponding to the shapes of the second substrate 14 and the partition wall 16, is disposed in a mold 64. Carbon black-containing polydimethylsiloxane resin is poured into the mold 64. The polydimethylsiloxane resin poured into the mold 64 is cured to integrally form the second substrate 14 and the partition wall 16. The die 62 is fabricated by subjecting a silicone substrate to photolithography. Note that, in the following, the member obtained by integrally forming the second substrate 14 and the partition wall 16 may be referred to as “the second substrate 14 having the partition wall 16.”

Returning to FIG. 5, in step S14, the openings 26 are formed by using a jig to open through-holes at predetermined positions of the second substrate 14 having the partition wall 16.

In step S16, the first substrate 12 is disposed on the partition wall 16 and, then, the first substrate 12 is pressed against the partition wall 16 to join the partition wall 16 to the first substrate 12. As a result, the first microchannels 22 and the second microchannels 24 are formed from the first substrate 12, the second substrate 14, and the partition wall 16.

In step S20, micropipettes are used to respectively fill the first microchannels 22 with the calibration curve liquids CCL1 to CCL5 through the openings 26. In one example, the calibration curve liquids CCL1 to CCL5 are prepared by diluting, in a stepwise manner, a standard liquid including the measurement target substance Ag1. Note that the competitive reaction of the measurement target substance Ag1 and the fluorescent-labeling derivative AgF1 against the antibody Abl in the calibration curve liquids CCL reaches equilibrium.

In step S30, the adhesive layer 34 of the sealing member 30 is affixed to the first main surface 14a of the second substrate 14, thereby closing the openings 26 of the first microchannels 22 by the sealing member 30 to seal the calibration curve liquids CCL1 to CCL5. In the present embodiment, the openings 26 of the second microchannels 24 are also closed by the sealing member 30. The microdevice 10 can be fabricated as described above. The fabricated microdevice 10 is stored at a low temperature (for example, 5° C. or lower).

Next, an immunoassay method of the measurement target substance Ag1 (that is, detection of the measurement target substance Ag1) using the microdevice 10 is described. Firstly, an analysis device 100 that detects the measurement target substance Ag1 is described.

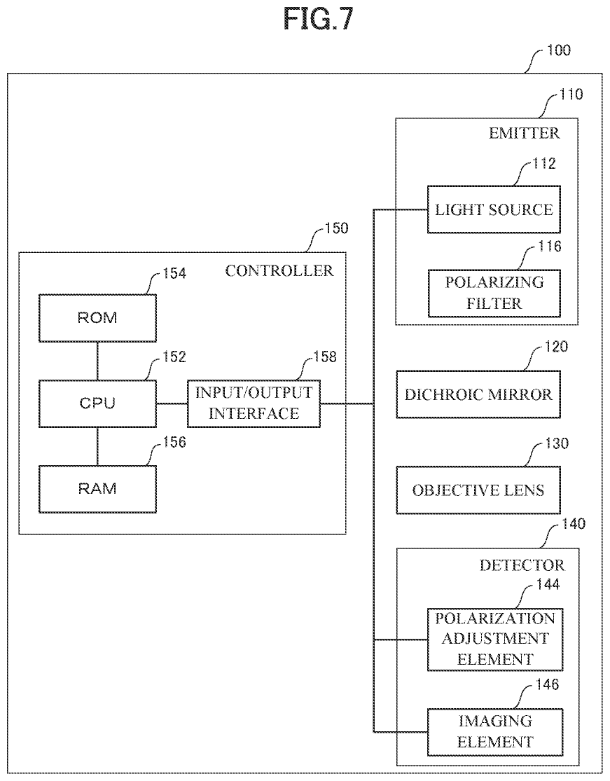

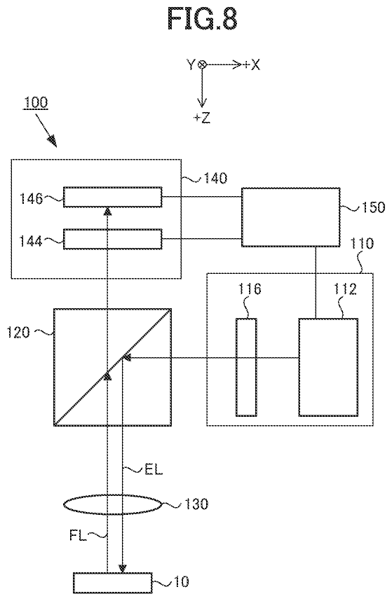

As illustrated in FIGS. 7 and 8, the analysis device 100 includes an emitter 110, a dichroic mirror 120, an objective lens 130, a detector 140, and a controller 150.

As illustrated in FIG. 8, the emitter 110 of the analysis device 100 emits linearly polarized excitation light EL in the −X direction. As illustrated in FIGS. 7 and 8, the emitter 110 includes a light source 112, a polarizing filter 116, and non-illustrated optical components such as an excitation light filter, a condenser lens, and the like. The light source 112 emits the excitation light EL in the −X direction. In one example, the light source 112 is constituted by an LED element. The polarizing filter 116 converts the excitation light EL to linearly polarized light.

As illustrated in FIG. 8, the dichroic mirror 120 of the analysis device 100 reflects the linearly polarized excitation light EL emitted from the emitter 110 in the direction (the +Z direction) in which the microdevice 10 is disposed. Additionally, the dichroic mirror 120 transmits the fluorescence FL emitted from the microdevice 10.

The microdevice 10 is disposed on the +Z side of the dichroic mirror 120, with the first substrate 12 facing the −Z direction. The linearly polarized excitation light EL reflected by the dichroic mirror 120 enters the microchannels 20 from the first substrate 12 of the microdevice 10. The microdevice 10 emits the fluorescence FL in the −Z direction.

As illustrated in FIG. 8, the objective lens 130 of the analysis device 100 is disposed between the dichroic mirror 120 and the microdevice 10. The objective lens 130 condenses the excitation light EL and the fluorescence FL.

As illustrated in FIG. 8, the detector 140 of the analysis device 100 is disposed on the −Z side of the dichroic mirror 120. The detector 140 detects the fluorescence FL emitted from the microdevice 10. As illustrated in FIGS. 7 and 8, the detector 140 includes a polarization adjustment element 144, an imaging element 146, and non-illustrated optical components such as an absorption filter, an imaging lens, and the like. The polarization adjustment element 144 adjusts the polarization direction of the fluorescence FL. The polarization adjustment element 144 adjusts the polarization direction of the fluorescence FL to a direction parallel to the polarization direction of the excitation light EL emitted from the emitter 110, and a direction perpendicular to the polarization direction of the excitation light EL emitted from the emitter 110. In one example, the polarization adjustment element 144 is a liquid crystal element. The imaging element 146 detects, as an image, the fluorescence FL emitted from the polarization adjustment element 144. In one example, the imaging element 146 is a complementary metal oxide semiconductor (CMOS) image sensor.

The controller 150 of the analysis device 100 controls the emitter 110 and the detector 140. Additionally, the controller 150 calculates, from the image of the fluorescence FL detected by the imaging element 146, the degree of polarization P of the fluorescence FL emitted from the calibration curve liquids CCL that the first microchannels 22 are filled with and the measurement target liquid MTL that the second microchannels 24 are filled with. The controller 150 creates, from the degree of polarization P of the fluorescence FL emitted from the calibration curve liquids CCL1 to CCL5 and the concentration of the measurement target substance Ag1 in the calibration curve liquids CCL1 to CCL5, a calibration curve of the degree of polarization P and the concentration of the measurement target substance Ag1. Furthermore, the controller 150 calculates the concentration of the measurement target substance Ag1 in the measurement target liquid MTL from the degree of polarization P of the fluorescence FL emitted from the measurement target liquid MTL and the created calibration curve.

The controller 150 includes a central processing unit (CPU) 152 that executes various processing, a read only memory (ROM) 154 that stores programs and data, a random access memory (RAM) 156 that stores data, and an input/output interface 158 that inputs and outputs signals to and from the various components. The CPU 152 executes the programs stored in the ROM 154 to realize the functions of the controller 150. The input/output interface 158 inputs and outputs signals to and from the CPU 152, and the emitter 110 and the detector 140.

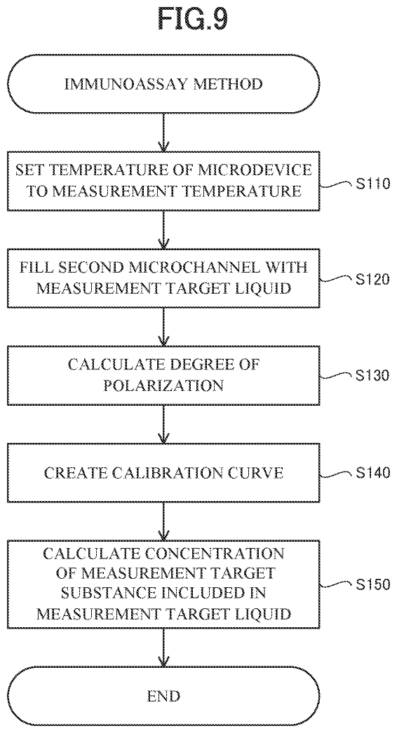

Next, an immunoassay method using the microdevice 10 is described. FIG. 9 is a flowchart illustrating the immunoassay method. The immunoassay method includes a step of setting the temperature of the microdevice 10 stored at a temperature of 5° C. or lower to a predetermined measurement temperature (step S110); and a step of filling the second microchannels 24 of the microdevice 10 with the measurement target liquid MTL (step S120). Furthermore, the immunoassay method includes a step of calculating the degree of polarization P of the fluorescence FL emitted from calibration curve liquids CCL1 to CCL5 and the measurement target liquid MTL (step S130); a step of creating a calibration curve of the degree of polarization P and the concentration of the measurement target substance Ag1 from the calculated degree of polarization P of the fluorescence FL emitted from the calibration curve liquids CCL1 to CCL5 (step S140); and a step of calculating the concentration of the measurement target substance Ag1 included in the measurement target liquid MTL from the calculated degree of polarization P of the fluorescence FL emitted from the measurement target liquid MTL and the created calibration curve (step S150).

In step S110, the temperature of the microdevice 10 that is stored at a temperature of 5° or lower is set to a measurement temperature at which the degree of polarization P is to be measured. In one example, the measurement temperature is 20° C.

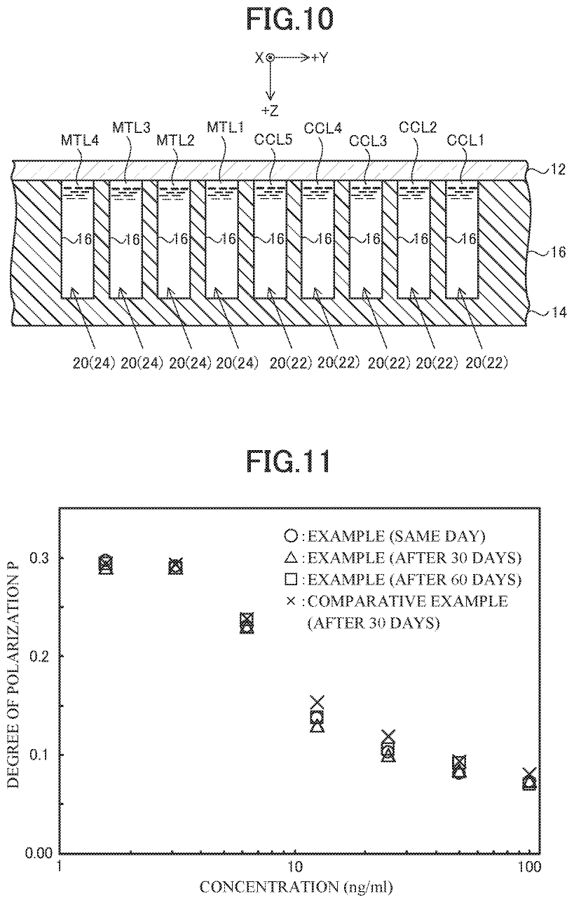

In step S120, firstly, a liquid including the measurement target substance Ag1 is added to a liquid including the same concentrations of the antibody Abl and the fluorescent-labeling derivative AgF1 as in the calibration curve liquid CCL. Thus, the measurement target liquid MTL is prepared. Next, after the competitive reaction of the measurement target substance Ag1 and the fluorescent-labeling derivative AgF1 against the antibody Abl has reached equilibrium in the measurement target liquid MTL, the second microchannels 24 are filled with the measurement target liquid MTL. Specifically, the sealing member 30 of the microdevice 10 is peeled and, then, micropipettes are used to fill the second microchannels 24 with the measurement target liquid MTL. In the present embodiment, as illustrated in FIG. 10, the four second microchannels 24 are respectively filled with measurement target liquids MTL1 to MTL4.

In step S130, firstly, the microdevice 10, in which the sealing member 30 has been peeled and the measurement target liquids MTL1 to MTL4 are filled, is set in the analysis device 100. Next, the linearly polarized excitation light EL is emitted from the emitter 110 of the analysis device 100, and the linearly polarized excitation light EL is irradiated on the measurement region S of the microdevice 10. Then, the degree of polarization P of the fluorescence FL emitted from the calibration curve liquids CCL1 to CCL5 and the measurement target liquids MTL1 to MTL4 is calculated by the controller 150 of the analysis device 100 from the image of the fluorescence FL detected by the detector 140 of the analysis device 100.

In this case, the degree of polarization P of the fluorescence FL is expressed as P=(Ih−Iv)/(Ih+Iv), where Ih is the intensity of the fluorescence FL that has a polarization direction parallel to the polarization direction of the excitation light EL, and Iv is the intensity of the fluorescence FL that has a polarization direction perpendicular to the polarization direction of the excitation light EL The measurement target substance Ag1 and the fluorescent-labeling derivative AgF1 cause a competitive reaction with the antibody Abl. Therefore, as the concentration of the measurement target substance Ag1 increases, the fluorescent-labeling derivative AgF1 not bonded to the antibody Abl increases and the degree of polarization P of the fluorescence FL decreases.

In step S140, the controller 150 of the analysis device 100 creates a calibration curve of the degree of polarization P and the concentration of the measurement target substance Ag1. Specifically, the controller 150 fits the degree of polarization P of the fluorescence FL emitted from the calibration curve liquids CCL1 to CCL5 and the concentration of the measurement target substance Ag1 in the calibration curve liquids CCL1 to CCL5 to a logistic function to create a calibration curve of the degree of polarization P and the concentration of the measurement target substance Ag1. In this case, it is preferable that the determination coefficient of the fitting is greater than 0.99.

In step S150, the controller 150 of the analysis device 100 calculates the concentration of the measurement target substance Ag1 included in the measurement target liquids MTL1 to MTL4 from the degree of polarization P of the fluorescence FL emitted from the measurement target liquids MTL1 to MTL4 and the created calibration curve. Thus, it is possible to obtain the concentration of the measurement target substance Ag1 included in the measurement target liquids MTL1 to MTL4.

As described above, in the microdevice 10, the calibration curve liquids CCL with which the first microchannels 22 are filled are sealed by the sealing member 30 and, as such, changes over time of the calibration curve liquids CCL are suppressed and stable storage over an extended period of time of the calibration curve liquids CCL at a low temperature is possible. Due to this, it is possible to create the calibration curve and detect the measurement target substance Ag1 in the measurement target liquid MTL by simply filling the second microchannels 24 with the measurement target liquid MTL. Accordingly, by using the microdevice 10, an immunoassay that has high measurement reliability can be performed with less work. Furthermore, variation in the calibration curve liquids CCL, variation in the work performed by a user, and the like can be suppressed and measurement accuracy can be improved by preparing a large amount of the calibration curve liquids CCL and manufacturing a large quantity of the microdevice 10.

### Modified Examples

Embodiments have been described, but various modifications can be made to the present disclosure without departing from the spirit and scope of the present disclosure.

The first substrate 12 of the present embodiment is a quartz glass substrate, but a configuration is possible in which the first substrate 12 is formed from another material that transmits the excitation light EL and the fluorescence FL.

In the embodiment described above, the second substrate 14 and the partition wall 16 are integrally formed, but a configuration is possible in which the second substrate 14 and the partition wall 16 are separately formed. Additionally, in the embodiment described above, the second substrate 14 and the partition wall 16 are formed from carbon-black-containing polydimethylsiloxane, but a configuration is possible in which the second substrate 14 and the partition wall 16 are formed from another material. For example, the polydimethylsiloxane may contain ferric oxide instead of carbon black. Additionally, it is preferable that the partition wall 16 is water repellent. With such a configuration, the microdevice 10 can suppress changes over time of the calibration curve liquids CCL even more, and can store the calibration curve liquids CCL for a more extended period of time. For example, it is preferable that the partition wall 16 is formed from a silicone resin.

The width of the microchannels 20 is preferably 500 μm or less and more preferably 300 μm or less.

In the embodiment described above, the microdevice 10 includes five of the first microchannels 22 and four of the second microchannels 24, but it is sufficient that the number of the first microchannels 22 is a number that enables creation of the calibration curve. Additionally, it is sufficient that the microdevice 10 includes at least one second microchannel 24.

A configuration is possible in which the microdevice 10 includes a plurality of sealing members 30, and each of the plurality of the sealing members 30 closes each of the openings 26.

In the embodiment described above, the sealing member 30 closes the openings 26 of the first microchannels 22 and the second microchannels 24, but it is sufficient that the sealing member 30 closes the openings 26 of the first microchannels 22. It is not necessary that the sealing member 30 closes the openings 26 of the second microchannels 24.

The sealing member 30 of the embodiment described above includes the base material 32 and the adhesive layer 34, but a configuration is possible in which the sealing member 30 is formed from only the base material 32. For example, the sealing member 30 may be formed from a silicone resin that adheres to the second substrate 14.

It is preferable that the base material 32 is flexible. Such a configuration enables the sealing member 30 to adhere more closely to the second substrate 14, thereby more tightly closing the openings 26. Furthermore, it is preferable that the surface of the sealing member 30 that closes the openings 26 is water repellent. For example, it is preferable that the adhesive layer 34 of the sealing member 30 is water repellent. With such a configuration, the microdevice 10 can suppress changes over time of the calibration curve liquids CCL even more, and can store the calibration curve liquids CCL for a more extended period of time.

A configuration is possible in which the calibration curve liquids CCL include at least one of zinc sulphate and sodium azide as a preservative.

In the creation of the calibration curve, the function that is fitted to is not limited to a logistic function. For example, the function that is fitted to may be a Boltzmann function, a Sigmoid Weibull function, or the like.

In the manufacturing method for the microdevice, a configuration is possible in which, after step S30, a step is executed for measuring the degree of polarization P of the sealed calibration curve liquids CCL to create the calibration curve of the degree of polarization P and the concentration of the measurement target substance Ag1. Due to this configuration, the accuracy of the calibration curve can be confirmed in advance. In this case, it is preferable that the determination coefficient of the fitting is greater than 0.99.

Preferred embodiments of the present disclosure have been described, but the present disclosure should not be construed as being limited to these specific embodiments. The scope of the invention is defined only by the included claims, along with the full range of equivalents to which such claims are entitled.

### Examples

Hereinafter, the present disclosure is described in detail using examples, but the present disclosure is not limited to these examples.

A microdevice 10 was prepared in which seven of the nine microchannels 20 were respectively filled with seven calibration curve liquids CCL. The degree of polarization P of the fluorescence FL emitted from the seven calibration curve liquids CCL on the fabrication date of the microdevice 10 and after storing the microdevice 10 at 5° C. for 30 days and 60 days was measured using the analysis device 100.

Additionally, each of the seven calibration curve liquids CCL was stored in a polypropylene microtube (diameter: 5 mm) at 5° C. Then, after 30 days, the empty microchannels 20 of the microdevice 10 were filled with each of the stored seven calibration curve liquids CCL, and the analysis device 100 was used to measure, as a comparative example, the degree of polarization P of the fluorescence FL emitted from the stored seven calibration curve liquids CCL.

The width of the microchannels 20 of the fabricated microdevice 10 is 200 μm. A flexible film including a silicone resin base material 32 and a silicone adhesive layer (the adhesive layer 34) was used as the sealing member 30. The seven calibration curve liquids CCL were prepared using a commercially available prostaglandin E2 measurement kit. Immediately after the seven calibration curve liquids CCL were prepared, the microchannels 20 were respectively filled with the seven calibration curve liquids CCL, and the seven calibration curve liquids CCL were also stored in microtubes. The concentration of the prostaglandin E2 in each of the seven calibration curve liquids CCL was 100 ng/ml, 50 ng/ml, 25 ng/ml, 12.5 ng/ml, 6.25 ng/ml, 3.125 ng/ml, and 1.5625 ng/ml. Note that the remaining one microchannel 20 was filled with phosphate buffered saline (PBS).

FIG. 11 illustrates the relationship between the concentration of the prostaglandin E2 and the degree of polarization P measured in the examples and the comparative example. In the examples, there was hardly any change over time of the degree of polarization P, even after storing at 5° C. for 60 days. However, in the comparative example, the degree of polarization P particularly increased at the concentrations of 25 ng/ml and 12.5 ng/ml, and changes over time of the degree of polarization P occurred. Accordingly, closing the openings 26 of the first microchannels 22 by the sealing member 30 to seal the calibration curve liquids CCL, enables the suppression of changes over time of the calibration curve liquids CCL and the stable storage of the calibration curve liquids CCL for an extended period of time.

The foregoing describes some example embodiments for explanatory purposes.

Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A microdevice, comprising:
a plurality of calibration curve liquids including a measurement target substance of a predetermined concentration, the predetermined concentration in each of the plurality of calibration curve liquids being mutually different, an antibody that specifically binds to the measurement target substance, and a fluorescent-labeling derivative that fluorescently labels the measurement target substance and competes with the measurement target substance to specifically bind to the antibody;
a plurality of first microchannels respectively filled with the plurality of calibration curve liquids;
at least one second microchannel to be filled with a measurement target liquid including an unknown concentration of the measurement target substance, the antibody, and the fluorescent-labeling derivative; and
a sealing member that closes an opening of the first microchannels to seal the calibration curve liquids.

2. The microdevice according to claim 1, wherein the sealing member is water repellent.

3. The microdevice according to claim 1, wherein the sealing member includes a base material that is flexible and an adhesive layer that is water repellent.

4. The microdevice according to claim 1, wherein the calibration curve liquids include at least one of zinc sulphate and sodium azide.

5. The microdevice according to claim 1, wherein a determination coefficient of a calibration curve of a degree of polarization and the concentration of the measurement target substance, obtained by measuring a degree of polarization of the sealed plurality of calibration curve liquids, is greater than 0.99.

6. A manufacturing method for a microdevice, the method comprising:
forming a plurality of first microchannels, and at least one second microchannel to be filled with a measurement target liquid including an unknown concentration of a measurement target substance, an antibody that specifically binds to the measurement target substance, and a fluorescent-labeling derivative that fluorescently labels the measurement target substance and competes with the measurement target substance to specifically bind to the antibody;
filling the plurality of first microchannels respectively with a plurality of calibration curve liquids including the measurement target substance of a predetermined concentration, the predetermined concentration in each of the plurality of calibration curve liquids being mutually different, the antibody, and the fluorescent-labeling derivative; and
sealing the plurality of calibration curve liquids by closing, by a sealing member, an opening of the first microchannels that are filled with the calibration curve liquids.

7. The manufacturing method for a microdevice according to claim 6, further comprising:
creating a calibration curve of a degree of polarization and the concentration of the measurement target substance by measuring the degree of polarization of the sealed plurality of calibration curve liquids.

8. An immunoassay method, comprising:
setting a temperature of the microdevice according to claim 1, that is stored at a temperature of 5° C. or lower, to a predetermined measurement temperature;
filling the second microchannel with the measurement target liquid;
calculating a degree of polarization of fluorescence emitted from the plurality of calibration curve liquids and the measurement target liquid with which the second microchannel is filled;
creating a calibration curve of the degree of polarization and a concentration of the measurement target substance from the calculated degree of polarization of the fluorescence emitted from the plurality of calibration curve liquids; and
calculating the concentration of the measurement target substance included in the measurement target liquid from the calculated degree of polarization of the fluorescence emitted from the measurement target liquid and the created calibration curve.
